(19) United States
(12) Patent Application Publication
(10) Pub. No.: US 2025/0043107 A1
(43) Pub. Date: Feb. 6, 2025

(54) BIO-BASED PLASTICIZERS

(71) Applicant: CARGILL, INCORPORATED, Wayzata, MN (US)

(72) Inventors: William BRAGG, Wayzata, MN (US); Xiangfu SHI, Medina, MN (US); Christopher Patrick STEVERMER, St. Louis Park, MN (US)

(73) Assignee: CARGILL, INCORPORATED, Wayzata, MN (US)

(21) Appl. No.: 18/718,136

(22) PCT Filed: Dec. 14, 2022
(86) PCT No.: PCT/US2022/081518

§ 371 Date: Jun. 10, 2024

Related U.S. Application Data
(60) Provisional application No. 63/265,394, filed on Dec. 14, 2021.

(51) Int. Cl. C08K 5/1515; C08J 3/18

(52) U.S. Cl. CPC C08K 5/1515; C08J 3/18; C08J 2327/06; C08J 2367/04; C08K 2201/019

(57) ABSTRACT

The disclosure relates to a plasticizer composition comprising an epoxidized fatty acid C1-C6 alkyl ester component wherein the epoxidized fatty acid C1-C6 alkyl ester component contains (i) less than 6% saturated fatty acid residues and (ii) less than 2% monoacylglycerols. The disclosure further relates to a plasticized polymer composition, comprising (a) 100 parts by weight of at least one polymer resin; and (b) about 10 to 150 parts by weight of a plasticizer compounded with the at least one polymer resin, wherein the plasticizer comprises an epoxidized fatty acid C1-C6 alkyl ester component wherein the epoxidized fatty acid C1-C6 alkyl ester component contains (i) less than 7% saturated fatty acid residues and (ii) less than 2% monoacylglycerols.

### CROSS-REFERENCE TO RELATED APPLICATIONS

This application claims the benefit of U.S. Provisional Application No. 63/265,394, filed Dec. 14, 2021, which is incorporated by reference herein in its entirety.

### TECHNICAL FIELD

This application relates to plasticizer derived from natural sources. Specifically, epoxidized fatty acid alkyl esters with low saturated fatty acid content and low MAG content.

### BACKGROUND

By definition, plasticizers are compounds or chemical substances added to a polymer matrix in order to impart flexibility, processability-workability, lubricity, softness, or elasticity.

Plasticizers are used in elastomers and plastics articles, such as in PVC (Polyvinyl Chloride), EVA (Ethylene vinyl acetate) and PU (Polyurethanes), among others, in many application materials as adhesives and sealants, films and sheets, flooring and wall covering, laminates, hoses, footwear, coated fabric materials (i.e., synthetic leathers and tarpaulins), childcare articles (i.e., toys), wires and cables, medical devices (e.g., tubing, bags) among others.

The choice of a plasticizer is an important factor in the formulation. When selecting a product, the formulator considers the significant qualities of the plasticizer, such as compatibility, permanence, efficiency and, of course, cost.

The use of plasticizers allows the manufacturer of vinyl compounds to balance the multiple required characteristics of its compound. Originally, plasticizers were used to transform rigid polyvinyl chloride (PVC) resins into more flexible products.

PVC products are divided into two main classes: rigid and flexible. Plasticizers have been used for many years in the production of flexible PVC for a wide variety of applications. The level and type of plasticizer used is selected to obtain the characteristics required for each application. They are generally colorless and odorless, relatively non-volatile liquids and exhibiting low solubility in water. They are mostly esters or polyesters, of acids such as adipic, phosphoric, sebaceous, trimetylic, or azelatic acids.

The plasticizers that have high compatibility with PVC are regarded as primary plasticizers and those with limited compatibility, as secondary plasticizers. Secondary plasticizers are used as co-additives to complement a specific material performance, for instance, epoxidized soybean oil (ESO) is largely used as secondary plasticizer to impart improved thermal stability for the PVC articles.

Plasticizers are divided according to their chemical characteristics: Phthalate: DIBP (di iso butyl phthalate), DOP (dioctyl phthalate), DIDP (di iso decyl phthalate), DINP (diisononyl phthalate), DEHP (di(2-etiyhexyl) phthalate); Adipates: DOA (dioctyl adipate); Azelates: DOZ (dioctyl azelate); Triesters: TOTM (trioctyl trimellitate); Polyesters: polymeric plasticizers; Epoxidized: ESO (epoxidized soybean oil); Phosphate: TCP (tricresyl phosphate); and Dioctyl cyclohexanoate (DOCH). The phthalic plasticizers (DOP, DIBP, DIDP, and DINP) are the traditional and most widely used because of the favorable cost and performance balance.

The secondary plasticizer most used is ESO, which also acts as auxiliary in the thermal stabilization of PVC together with stabilizers based on salts of barium, calcium, and zinc.

Polyvinyl chloride is one of the most consumed plastics. For PVC, the phthalate plasticizers are still the most commonly used, and of these, dioctyl phthalate (DOP) and the diisononyl phthalate (DINP) have been considered the standard and used for multi-purpose PVC plasticizers. DOP (di octyl phthalate) is used in blood bags, human tissue simulators, packaging, shoes, tubes and profiles, and represents more than 50% of the total plasticizers produced in the world.

An additional common use of plasticizers is in production of plastisol. Plastisol is a suspension of PVC or other polymer particles in a liquid plasticizer. Aside from molding, plastisol is also used in specific manufacturing process commonly used for the production of synthetic leather, tarpaulins, coated fabric articles, flooring and toys, among others.

Despite all the positive aspects, phthalate plasticizers have undergone extensive testing to evaluate possible health and environmental effects. To date, four classified low orthophthalates—DOP, DBP, DIBP and BBP—have been found to have adverse endocrine-related effects in laboratory animal studies with specific thresholds.

In some cases, phthalates have been shown to be extracted from the polymeric matrix when in contact with certain material. This effect makes them impossible to use in some situations, especially when the PVC material is in direct contact with food. Overall, the regulatory agencies, especially in Europe and North America, have limited the use of these substances in articles which will potentially be in contact with skin or food for prolonged time skin and for childcare articles.

There are four (4) main forms of loss of plasticizer from a plasticized polymer: (i) volatilization: loss of plasticizer from the material to the atmosphere; (ii) exudation: loss of plasticizer by emission from the material; (iii) migration: loss of plasticizer by transfer between two surfaces that are in contact; (iv) extraction: loss of plasticizer from the material for liquids (oils, water, greases among other agents).

Accordingly, the plasticizer market today faces many regulatory issues, mainly the limited use of the phthalates. The restrictions limit use in childcare articles, materials which may have prolonged contact with the human skin, and contact with food.

Additionally, recently, the adipose tissue has been recognized as a true endocrine organ, and a subset of EDCs have been named Metabolism Disrupting Chemicals (MDCs) because of their ability to promote adiposity and alteration of energy homeostasis. MDCs include plasticizers such as bisphenol (BPA) and certain phthalates used in PVC plastics, in outdoor applications (roofs, furniture) and dip-coating.

With that, it has been an increasing demand and interest by the market and consumers looking for alternative plasticizers which can replace the phthalates and be compliant with the global regulatory legislation. The so-called biobased plasticizers, made from modified natural based feedstocks such as vegetable oils, fatty acids and their derivatives, have been identified as feasible options and viable solutions to help the phasing out of these regulated petro-based substances.

### SUMMARY

In view of the issues involving the phthalate compounds and currently known alternative plasticizers, there is a need for an effective plasticizer developed from renewable sources (i.e., “green”) with good performance that is not based on phthalate.

Epoxidized natural oils and fatty acid alkyl esters are known in the prior art as plasticizers. A wide variety of derivatives of epoxidized oils have been investigated to this end. These renewable alternatives to known chemical plasticizers have not fully succeeded in matching the performance of phthalates and still suffer from significant drawbacks. These natural oil-based materials are often criticized in the art as lacking compatibility in PVC formulations and suffering from exudation and release of volatile organic components (VOCs). These perceived shortcomings have led those in the art to increasingly complex solutions to this overcome this unmet need. These complex solutions come with increased cost and complicated manufacturing.

The present disclosure provides an elegant solution to overcome the performance shortcomings recognized in the art. The inventors have surprisingly discovered that the performance of epoxidized fatty acid alkyl esters can be greatly improved by carefully managing the presence of particular impurities.

When epoxidized soybean oil, for example, is transesterifed with methanol to create epoxidized fatty acid methyl esters (also referred to as EFAME or epoxidized methylsoyate), there is a significant amount of monoacylglycerides (MAGs) created in the process and remaining in the product. Samples can easily have 2 or more percent of MAGs. The Applicants have discovered that MAG content in a plasticizer at this level will contribute to crystallization or fogging of plasticized PVC articles that are exposed to cold temperatures. This crystallization or fogging of the PCV article renders them unfit for consumers or manufacturers.

In addition, natural oils used to prepare fatty acid alkyl esters contain significant amounts of saturated fatty acid residues. These fatty acids cannot be epoxidized and are less compatible with the PVC matrix resulting in exudation. Natural oils contain significant amounts of saturated fatty acids, in case of soybean oil, up to 14-16% saturates. The applicants have shown that such high levels of saturated fatty acid esters in the plasticizer contribute to mass loss through VOCs in plasticized PVC products. This exudation and VOC property restricts utilization of these materials by manufacturers. Accordingly, increasingly complex and expensive solutions have been sought in the industry.

Additionally, it has been found that plasticizers as described herein can exhibit differentiated and significant improvement in performance parameters measured by the hardness, density, and mass loss in dry-blend formats for use in suspension PVC applications and/or in emulsions for use in plastisol PVC applications.

As the present application discloses, saturated fatty acid esters are not effective plasticizers. They carry no epoxide groups and are a detriment to plasticizer formulations. Compositions of the present invention have reduced levels of saturated fatty acid esters and hence are more effective than epoxidized esters from standard vegetable oils. The higher effectiveness of the present disclosure's compositions makes them more efficient, meaning the same level of plasticizing affect can be achieved with lower amounts of plasticizers. This allows PVC manufacturers to blend in lower cost fillers to achieve comparable PVC products with a lower cost.

It further has been found that plasticizers as described herein can exhibit excellent low VOC and exudation properties, even in final applications comprising greater than 50 PHR, or greater than 70 PHR, or greater than 80 PHR levels of inclusion.

These beneficial properties are achieved in a plasticizer composition comprising high content of renewable sourced ingredients. The source ingredients for the present plasticizer composition are readily available, and the resulting plasticizer composition may be prepared at a cost competitive price.

Accordingly, an aspect of the present disclosure is a plasticizer composition comprising an epoxidized fatty acid C1-C6 alkyl ester component wherein the epoxidized fatty acid C1-C6 alkyl ester component comprises (i) less than 6% saturated fatty acid residues, and (ii) less than 2% monoacylglycerols.

A further aspect is a plasticized polymer composition, comprising (a) 100 parts by weight of at least one polymer resin; and (b) about 10 to 150 parts by weight of a plasticizer compounded with the at least one polymer resin, wherein the plasticizer comprises an epoxidized fatty acid C1-C6 alkyl ester component wherein the epoxidized fatty acid C1-C6 alkyl ester component contains (i) less than 7% saturated fatty acid residues and (ii) less than 2% monoacylglycerols.

A further aspect is a process to prepare a plasticizer comprising the steps of a) providing a fatty acid C1-C6 alkyl ester component having less than 2% monoacylglycerols; b) epoxidizing the fatty acid C1-C6 alkyl ester component to yield an epoxidized fatty acid C1-C6 alkyl ester component; and c) removing the saturated fatty acid C1-C6 alkyl esters in the epoxidized fatty acid C1-C6 alkyl ester component to level below 7%, and d) recovering the plasticizer.

### DETAILED DESCRIPTION

Reference will now be made in detail to certain aspects of the disclosed subject matter. While the disclosed subject matter will be described in conjunction with the enumerated claims, it will be understood that the exemplified subject matter is not intended to limit the claims to the disclosed subject matter. One aspect described in conjunction with a particular embodiment is not necessarily limited to that embodiment and can be practiced with any other embodiment(s).

Throughout this document, values expressed in a range format should be interpreted in a flexible manner to include not only the numerical values explicitly recited as the limits of the range, but also to include all the individual numerical values or sub-ranges encompassed within that range as if each numerical value and sub-range is explicitly recited. For example, a range of “about 0.1% to about 5%” or “about 0.1% to 5%” should be interpreted to include not just about 0.1% to about 5%, but also the individual values (e.g., 1%, 2%, 3%, and 4%) and the sub-ranges (e.g., 0.1% to 0.5%, 1.1% to 2.2%, 3.3% to 4.4%) within the indicated range. The statement “about X to Y” has the same meaning as “about X to about Y,” unless indicated otherwise. Likewise, the statement “about X, Y, or about Z” has the same meaning as “about X, about Y, or about Z,” unless indicated otherwise.

As used herein, the singular forms “a,” “an,” and “the” and similar referents in the context of describing the elements (especially in the context of the following claims) include plural referents unless the context clearly dictates otherwise. For example, reference to “a substituent” encompasses a single substituent as well as two or more substituents, and the like. It is understood that any term in the singular may include its plural counterpart and vice versa, unless otherwise indicated herein or clearly contradicted by context

As used herein, the following terms have the following meanings unless expressly stated to the contrary.

The term “or” is used to refer to a nonexclusive “or” unless otherwise indicated. The statement “at least one of A and B” has the same meaning as “A, B, or A and B.”

In addition, it is to be understood that the phraseology or terminology employed herein, and not otherwise defined, is for the purpose of description only and not of limitation. Any use of section headings is intended to aid reading of the document and is not to be interpreted as limiting; information that is relevant to a section heading may occur within or outside of that particular section. Any publications, patents, and patent documents referred to in this document are incorporated by reference herein in their entirety, as though individually incorporated by reference. In the event of inconsistent usages between this document and those documents so incorporated by reference, the usage in the incorporated reference should be considered supplementary to that of this document; for irreconcilable inconsistencies, the usage in this document controls.

As used herein, the terms “for example,” “for instance,” “such as,” or “including” are meant to introduce examples that further clarify more general subject matter. Unless otherwise specified, these examples are provided only as an aid for understanding the applications illustrated in the present disclosure and are not meant to be limiting in any fashion.

In the methods described herein, the acts can be carried out in any order without departing from the principles of the disclosure, except when a temporal or operational sequence is explicitly recited. Furthermore, specified acts can be carried out concurrently unless explicit claim language recites that they be carried out separately. For example, a claimed act of doing X and a claimed act of doing Y can be conducted simultaneously within a single operation, and the resulting process will fall within the literal scope of the claimed process.

The term “about” as used herein can allow for a degree of variability in a value or range, for example, plus or minus within 10%, within 5%, or within 1% of a stated value or of a stated limit of a range and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%.

The term “PHR” means parts per 100 parts of resin as measured by weight. It is a well-known measurement of inclusion by weight of components into a PVC or plasticized composition relative to the amount of resin.

As used herein, the term “natural oil” may refer to oil derived from plants or animal sources. The term “natural oil” includes triglyceride containing natural oil derivatives, unless otherwise indicated. Examples of natural oils include, but are not limited to, vegetable oils, algae oils, animal fats, and derivatives of these oils, combinations of any of these oils, and the like. Representative non-limiting examples of vegetable oils include canola oil, rapeseed oil, coconut oil, corn oil, cottonseed oil, olive oil, palm oil, peanut oil, safflower oil, sesame oil, soybean oil, sunflower oil, linseed oil, palm kernel oil, tung oil, jatropha oil, mustard oil, camelina oil, pennycress oil, hemp oil, algal oil, and castor oil. Representative non-limiting examples of animal fats include lard, tallow, poultry fat, yellow grease, and fish oil. In some aspects, the natural oil may be refined, bleached, and/or deodorized. In some aspects, the natural oil is present individually or as mixtures thereof.

A “monoacylglyceride” refers to a molecule having a glycerol moiety with a single fatty acid residue that is linked via an ester bond. The terms “monoacylglycerol,” “monoacylglyceride,” “monoglyceride,” and “MAG” are used interchangeably herein. Monoacylglycerides include 2-acylglycerides and 1-acylglycerides.

A “diacylglyceride” refers to a molecule having a glycerol moiety having two fatty acid residues linked via ester bonds. The terms “diacylglycerol,” “diacylglyceride,” “diglyceride,” and “DAG” are used interchangeably herein. Diacylglycerides include 1,2-diacylglycerides and 1,3-diacylglycerides.

A “triacylglyceride” refers to a molecule having a glycerol moiety that is linked to three fatty acid residues via ester bonds. The terms “triacylglycerol,” “triacylglyceride,” “triglyceride,” and “TAG” are used interchangeably herein.

The term “fatty acid” as used herein can refer to a molecule comprising a hydrocarbon chain and a terminal carboxylic acid group. As used herein, the carboxylic acid group of the fatty acid may be modified or esterified, for example as occurs when the fatty acid is incorporated into a glyceride or another molecule (e.g., COOR, where R refers to, for example, a carbon atom). Alternatively, the carboxylic acid group may be in the free fatty acid or salt form (i.e., COO″ or COOH). The ‘tail’ or hydrocarbon chain of a fatty acid may also be referred to as a fatty acid chain, fatty acid sidechain, or fatty chain. The hydrocarbon chain of a fatty acid will typically be a saturated or unsaturated aliphatic group. A fatty acid having N number of carbons, will typically have a fatty acid side chain having N−1 carbons. However, the subject application also relates to modified forms of fatty acids and thus the term fatty acid may be used in a context in which the fatty acid has been substituted or otherwise modified as described. For example, in various aspects, a fatty acid may be substituted with another alkyl chain as is the case for isostearic acid or a hydroxy group as is the case with castor oil.

An “acylglyceride” refers to a molecule having at least one glycerol moiety with at least one fatty acid residue that is linked via an ester bond. For example, acylglycerides can include monoacylglycerides, diacylglycerides, triacylglycerides. The group acylglycerides can be further refined by additional descriptive terms and can be modified to expressly exclude or include certain subsets of acylglycerides. For example, the phrase mono- and di-acylglycerides refers to MAGs (monoacylglycerides) and DAGs (diacylglycerides), while the phrase non-MAG/non-DAG acylglycerides refers to a group of acylglycerides which exclude MAGs and DAGs.

A “fatty acid residue” is a fatty acid in its acyl or esterified form.

The levels of particular types of fatty acids may be provided herein in percentages out of the total fatty acid content of an oil. Unless specifically noted otherwise, such percentages are weight percentages based on the total fatty acids, including free fatty acids and esterified fatty acids as calculated experimentally.

A “saturated” fatty acid is a fatty acid that does not contain any carbon-carbon double bonds in the hydrocarbon chain. An “unsaturated” fatty acid contains one or more carbon-carbon double bonds. A “polyunsaturated” fatty acid contains more than one such carbon-carbon double bond while a “monounsaturated” fatty acid contains only one carbon-carbon double bond. Carbon-carbon double bonds may be in one of two stereoconfigurations denoted cis and trans. Naturally-occurring unsaturated fatty acids are generally in the “cis” form.

Non-limiting examples of fatty acids include C8, C10, C12, C14, C16 (e.g., C16:0, C16:1), C18 (e.g., C18:0, C18:1, C18:2, C18:3, C18:4), C20 and C22 fatty acids. For example, the fatty acids can be caprylic (8:0), capric (10:0), lauric (12:0), myristic (14:0), palmitic (16:0), stearic or isostearic (18:0), oleic (18:1), linoleic (18:2) and linolenic (18:3) acids.

The term “C8-C18 fatty acid” means a fatty acid containing 8-18 carbons. The C8-C18 fatty acid may be straight or branched. In some aspects, the C8-C18 fatty acid is a C16 and/or C18 fatty acid.

The C8-C18 fatty acid may be a mixture of C8-C18 fatty acids.

The term “C1-C6 alkyl ester or alkyl ester” means a fatty acid containing 8-22 carbons which is esterified with a C1-C6 alkyl group. The C1-C6 alkyl group may be straight or branched. In some aspects, the C1-C6 alkyl has a straight chain. In some aspects, the C1-C6 alkyl is a methyl or ethyl group. In some aspects, the C1-C6 alkyl is a methyl group.

The fatty acid C1-C6 alkyl esters may be partially, substantially, or completely epoxidized. In one aspect, the epoxidized fatty acid C1-C6 alkyl esters are derived from a natural oil. In one aspect, the epoxidized fatty acid C1-C6 alkyl esters are epoxidized FAME from soybean oil. In another aspect the C1-C6 alkyl esters are epoxidized FAME from canola or rapeseed oil.

In some aspects, the fatty acid C1-C6 alkyl esters comprises less than about 2.5%, less than 2%, less than 1%, or less than 0.5% C20 or greater fatty acid residues. In some aspects, the fatty acid C1-C6 alkyl esters comprises between about 0.1% 2.5%, between about 0.1% and 2%, or between about 0.1% and 1% C20 or greater fatty acid residues. A more narrow range of fatty acids the plasticizing composition can assist in uniformity and lower variability in subsequent use.

The fatty acid composition of an oil can be determined by methods well known in the art. The American Oil Chemist's Society (AOCS) maintains analytical methods for a wide variety of tests performed on vegetable oils. Hydrolysis of the oil's components to produce free fatty acids, conversion of the free fatty acids to methyl esters, and analysis by gas-liquid chromatography (GLC) is the universally accepted standard method to determine the fatty acid composition of an oil sample. The AOCS Procedure Ce 1-62 describes the procedure used.

The terms “esterification or esterified” means the creation of an ester bond including: 1) the dehydration reaction of an alcohol with an acid; 2) transesterification, the reaction of an alcohol with an ester to form a new ester; or 3) interesterification, the rearrangement of fatty acids within a triacylglycerol structure.

The term “Shore Hardness” as used herein means hardness, or resistance to denting or deflecting, of a material upon the application of force. Shore Hardness is a well known parameter used to evaluate the mechanical properties of materials such as polymers, elastomers, and rubbers. Values are reported as “Shore A hardness” and higher numbers on the scale indicate a greater resistance to indentation and thus harder materials. Lower numbers indicate less resistance and softer materials. Shore Hardness can be evaluated by ASTM D2240 using the A scale. Test plaques are allowed 24 hours of rest before evaluation and value is reported after 1 second.

The term “Acid Value” (AV) as used herein is defined as the weight of KOH in mg needed to neutralize the organic acids present in 1 g of test sample and it is a measure of the free fatty acids present in the composition. AV can be determined by the AOCS Cd-3d-63.

The term “Hydroxyl Value” as used herein is defined as the hydroxyl value, expressed in milligrams of potassium hydroxide and corresponds to the number of hydroxyl groups present in 1 g of a sample, is one of the traditional characteristics of oils and fats. Hydroxyl Value may be determined by ASTM E1899-02.

The term “Iodine Value” (commonly abbreviated as IV) as used herein is the mass of iodine in grams that is consumed by 100 grams of a chemical substance. Iodine numbers are often used to determine the amount of unsaturation in fats, oils and waxes. In fatty acids, unsaturation occurs mainly as double bonds which are very reactive towards halogens, iodine in this case. Thus, the higher the iodine value, the more unsaturation is present in the sample. The Iodine Value of a material can be determined by the standard well-known Wijs method (A.O.C.S. Cd-1d-92).

The amount of MAG in compositions disclosed herein was determined using GC FID. The column: DB-5HT 30 m×320 um×0.25 um; injection volume was 1 uL; Injector temperature was 375° C.; Column pressure: was 15.858 psig; Total flow was 73 ml/min; Purge flow was 3 ml/min; Split Ratio was 20:1; Detector Temp was 375° C.; H2 flow was 35 ml/min; Air flow was 400 ml/min; Makeup Flow (N2) was 15 ml/min. Oven: 80° C. initial temp, equilibration time 2 minutes, hold time 5 minutes then ramp at 10° C./min to 350° C. and hold for 15 minutes for a total run time of 47 minutes. Sample preparation: 5 drops of sample in 0.5 ml pyridine, 0.5 ml BSTFA, warmed then diluted with ˜10 ml of toluene. Retention times: ˜8 min Glycerin; ˜16 min-methyl palmitate; ˜18 min methyl stearate; ˜19 min methyl mono-epoxy stearate; ˜20 min-methyl diepoxy stearate; ˜21 min-methyl triepoxy stearate; ˜24 min-Monoglyceride.

### Composition

In some aspects, the compositions described herein contain a low level of saturated fatty acids. In some aspects, the compositions contain less than 6% saturated fatty acids. In some aspects, the compositions contain less than 5% saturated fatty acids. In some aspects, the compositions contain less than 3% saturated fatty acids. In some aspects, the compositions contain between 1% and 6% saturated fatty acids. In some aspects, the compositions contain between 0.5%% and 5% saturated fatty acids. In some aspects, the compositions contain between 1% and 3% saturated fatty acids.

In some aspects, the composition may include minimal amount of monoglycerides. Preferably the composition would contain zero or a nondetectable amount of monoglycerides. Alternatively, the composition may include less than about 2 wt % of monoglycerides. In another aspect, the composition may include less than about 1 wt % of monoglycerides, or less than about 0.5 wt % of monoglycerides. In another aspect, the composition may include between about 0.1% to about 2 wt %; or between about 0.1% to about 1 wt %; or between about 0.1% to about 0.5 wt %; of monoglycerides.

In an aspect, the epoxidized fatty alkyl ester plasticizer composition or plasticized resin is substantially free of dioctyl phthalate (DOP), di-isononyl phthalate (DINP), dioctyl terephthalate (DOTP) and/or dioctyl cyclohexanoate (DOCH).

In an aspect, the composition is comprised of substantially an epoxidized fatty acid C1-C6 alkyl ester. In an aspect, the composition is comprised of substantially a mixture of epoxidized fatty acid C1-C6 alkyl esters. In an aspect, the composition is comprised of greater than 50% or greater than 90% epoxidized fatty acid C1-C6 alkyl esters. In an aspect, the composition is comprised of greater than 95% epoxidized fatty acid C1-C6 alkyl esters. In an aspect, the composition is comprised of between 90% and 98% epoxidized fatty acid C1-C6 alkyl esters. In an aspect, the composition is comprised of between 90% and 99.5% epoxidized fatty acid C1-C6 alkyl esters. In an aspect, the composition is comprised of between 95% and 98% epoxidized fatty acid C1-C6 alkyl esters.

In some aspects, the epoxidized fatty acid C1-C6 alkyl esters are derived from a triglyceride containing natural oil.

In some aspects, the natural oil is soybean oil, rapeseed oil, sunflower oil, or canola oil.

In some aspects, the epoxidized fatty acid C1-C6 alkyl esters are derived from soybean oil.

In some aspects, epoxidized fatty acid C1-C6 alkyl esters are not derived from tall oil.

In some aspects, the natural oil is selected from the group consisting of soybean oil, rapeseed oil, sunflower oil, and canola oil; but not tall oil.

In an aspect, the natural oil used in the present process has an Iodine Value of from about 80 to about 140 cg I/g.

Advantageously, the epoxidized fatty alkyl ester plasticizer composition has a favorably low APHA color value (sometimes referred to as the Hazen color value). For purposes of the present invention, the APHA color value is determined by the test described in ASTM D1209 and has the units “mg Pt/fl.” Alternatively, color may be measured using the Standard Test Method for Color of Transparent Liquids (Gardner Color Scale),” 2010, http://www.astm.org/Standards/D1544.htm.

In an aspect, the epoxidized fatty acid alkyl ester plasticizer composition has an APHA color value of less than about 250 mg Pt/fl. In an aspect, the epoxidized fatty acid alkyl ester plasticizer composition has an APHA color value of less than about 150 mg Pt/fl. In an aspect, the epoxidized fatty acid alkyl ester plasticizer composition has an APHA color value of from 10 to about 100 mg Pt/fl.

In an aspect, the epoxidized fatty acid alkyl ester plasticizer composition may comprise an additional component selected from any additive useful for plastic compositions, for instance stabilizers, antifogging agents, surfactants, biocides, fillers, slip agents, release agents, thickeners, lubricants, flow modifiers or processing aids, impact modifiers, pigments, viscosity reducers, flame retardants and diluent, or mixtures thereof.

Oxirane content of the epoxidized fatty acid alkyl esters depends on the degree of unsaturation in the starting natural oil used. In some aspects, the oxirane content is between about 6% and about 9%. Oxirane content can be easily determined by one of skill in the art. For example, using ASTM D1652-04.

### Method of Preparing Plasticizer Compositions

Epoxidized fatty acid C1-C6 alkyl esters can be prepared by two main methods that are known in the art. Both methods start with a natural oil as the base material. The oil can then be directly epoxidized to yield an epoxidized oil and the epoxidized oil then transesterified with a C1-C6 alcohol to yield an epoxidized fatty acid C1-C6 alkyl ester. Alternatively, the natural oil can first be transesterified and then epoxidized to yield the epoxidized fatty acid C1-C6 alkyl ester. The inventors have discovered that transesterifying an epoxidized natural oil, such as soybean oil, leads to a final product which contains unacceptably high levels of MAGs. These residual MAGs lead to poor plasticizer performance and unacceptable susceptibility for crystallization to occur in the final PVC products.

Alternatively, the careful preparation of C1-C6 alkyl esters from a natural oil with attention to driving the transesterification to completion can yield products with a low level of residual MAGs. Such a technique is well known in the arts for the preparation of bio-based fuels. This material can then be readily epoxidized by methods known in the art to yield epoxidized fatty acid esters having a reduced MAG content.

A reduced level of saturated fatty acid esters can be achieved by distilling or stripping the saturated fatty acid esters away from the epoxidized fatty acid alkyl esters. A sample of alkyl ester can be subjected to heat and reduced pressure to preferentially vaporize the unepoxidized saturated fatty acid alkyl esters. Such distillation procedures are well known in the art and include short path distillation (such as kugelrohr apparatus) or the use of a wiped film evaporator. These processes are performed at elevated temperature and at reduced pressure. A skilled person will appreciate that the effectiveness of the distillation or stripping will depend on the both the temperature utilized as well as the degree of vacuum applied. Typically, they can be performed at a temperature above 100° C. and a pressure below 0.5 torr.

Alternatively, a skilled artisan could start the production process with a fatty acid containing oil that is already low in saturates to avoid the need to remove saturated fatty acid esters later in the process.

A further aspect is a process to prepare a plasticizer comprising the steps of a) providing a fatty acid C1-C6 alkyl ester component having less than 2% monoacylglycerols; b) epoxidizing the fatty acid C1-C6 alkyl ester component to yield an epoxidized fatty acid C1-C6 alkyl ester component; and c) removing the saturated fatty acid C1-C6 alkyl esters in the epoxidized fatty acid C1-C6 alkyl ester component to level below 7%, and d) recovering the plasticizer.

### Method of Preparing PVC Compositions

The compositions of the invention can be considered bio-plasticizers and are “greener” and more environmentally friendly than the phthalates that they can replace. The compositions are useful for plasticizing thermoplastics or other polymers such as halogenated polymers, including polyvinyl chloride (PVC) homopolymers, PVC copolymers, and polyvinyl dichlorides and mixtures thereof. Useful co-monomers with vinyl chloride include, but are not limited to, vinyl acetate, vinyl alcohol, vinyl acetals, vinyl ethers, vinylidene chloride, lower alkyl (meth)acrylates, (meth)acrylic acid, lower alkyl olefins, vinyl aromatics such as styrene and styrene derivatives and vinyl esters and mixtures thereof. Chlorinated polyolefins, chlorinated rubbers, and/or acrylic acid functionalized polymers may also be plasticized using the composition of the invention. The composition may be used to soften other polymers, including but not limited to polyurethanes, polystyrene and its copolymers, polybutadiene copolymers, polyamides, rubber and/or synthetic rubbers, EPDM (synthetic rubber made from ethylene propylene diene monomer), and other similar polymers. Thermoset polymers may also be plasticized by the composition of the invention. In one embodiment, the epoxidized composition is used in biopolymers—such as polylactic acid, polyhydroxy butyrate, or polyamide 11 or mixtures thereof.

The epoxidized composition, in addition to its role as a plasticizer, also acts as an acid scavenger.

The composition may be used as the sole or primary plasticizer, or in combination with one or more other plasticizers, such as, but not limited to phthalates, and low and medium molecular weight polymeric plasticizers, or epoxidized natural oils

In some aspects, the plasticized polymer composition may further comprise an epoxidized natural oil. In some other aspects, the plasticized polymer composition may further not comprise an epoxidized natural oil.

The present invention is also directed to a plasticized polymer composition containing one or more polymers and a composition of the present invention where the composition is homogenously dispersed in the polymer composition. Such mixing or compounding is well known in the art. In some aspects, the composition is used as a plasticizer for the polymers, alone or in combination with other plasticizers. In other aspects, the polymers in the composition may be in the form of a polymer matrix. The polymer composition may further contain other additives known for use with polymers such as thermoplastics, including, but not limited to fillers, pigments, flame retardants, dyes, stabilizers, UV stabilizers, lubricants, surfactants, flow aids, and/or other plasticizers.

In an aspect, the epoxidized fatty acid alkyl ester plasticizer composition as described herein is mixed with an appropriate polymer resin, such as polyvinyl chloride (PVC), in an amount effective to provide a plasticizing effect. In an aspect, the epoxidized fatty acid alkyl ester plasticizer composition is provided in a dry-blend format for use in suspension PVC applications. In an aspect, the epoxidized fatty acid alkyl ester plasticizer composition is provided in a suspension for use in plastisol PVC applications. In an aspect, the epoxidized fatty acid alkyl ester plasticizer composition is mixed with an appropriate polymer resin at a mix ratio of from about 25 to about 300 PHR. In an aspect, the epoxidized fatty acid alkyl ester plasticizer composition is mixed with an appropriate polymer resin at a mix ratio of from about 10 to about 150 PHR. In an aspect, the epoxidized fatty acid alkyl ester plasticizer composition is mixed with an appropriate polymer resin at a mix ratio of from about 80 to about 150 PHR. In an aspect, the epoxidized fatty acid alkyl ester plasticizer composition is mixed with an appropriate polymer resin at a mix ratio of from about 60 to about 100 PHR. In an aspect, the polymer resin comprises PVC.

The final plasticized polymer product (e.g., PVC products) can be applied for the manufacturing or processing of flexible and semi-rigid polymer articles (e.g., PVC articles) for many industries, such as for footwear, childcare articles (i.e., toys), wires & cables, sealants & adhesives, coated fabric (i.e., synthetic leather and tarpaulins), flooring and medical devices (i.e., tubing and bags), among others.

A further aspect of the present invention includes a plasticized polymer composition, comprising (a) 100 parts by weight of at least one polymer resin; and (b) about 10 to 150 parts by weight of a plasticizer compounded with the at least one polymer resin, wherein the plasticizer comprises an epoxidized fatty acid C1-C6 alkyl ester component wherein the epoxidized fatty acid C1-C6 alkyl ester component contains (i) less than 7% saturated fatty acid residues and (ii) less than 2% monoacylglycerols.

A further aspect of the present invention includes a plasticized polyvinyl chloride composition, comprising (a) 100 parts by weight of at least one polyvinyl chloride resin; and (b) about 10 to 150 parts by weight of a plasticizer compounded with polyvinyl chloride resin, wherein the plasticizer comprises an epoxidized fatty acid C1-C6 alkyl ester component wherein the epoxidized fatty acid C1-C6 alkyl ester component contains; (i) less than 7% saturated fatty acid residues, and (ii) less than 2% monoacylglycerols.

A further aspect of the present invention includes a plasticized polyvinyl chloride composition, comprising (a) 100 parts by weight of at least one polyvinyl chloride resin; and (b) about 10 to 150 PHR of a plasticizer, wherein the plasticizer comprises an epoxidized fatty acid C1-C6 alkyl ester component wherein the epoxidized fatty acid C1-C6 alkyl ester component contains; (i) less than 7% saturated fatty acid residues, and (ii) less than 2% monoacylglycerols.

Any and every combination of two or more features disclosed herein for the compositions has been specifically contemplated and envisioned by the inventors. Therefore, the inventors have conceived of, and accordingly disclosed, every combination of single points and ranges disclosed for the level of saturated fatty acid residues, MAGs, hydroxyl value, type of natural oil, percentage of epoxidized fatty acid C1-C6 alkyl esters in the plasticizer composition, acid value, or iodine value.

### EXAMPLES

### Example 1

Four separate Example materials (1A-1D) were purchased or prepared to create the blending study. The study evaluated the effect of plasticizers containing varying levels of saturated fatty acid residues as well as the effect of varying concentrations of MAGs.

Example 1A (Low saturates—Low MAGs) (Saturates 2.4%-MAGs 0.4%) A plasticizer sample is prepared by the following procedure. 6050 g of Soy FAME (commercially available as methylsoyate from Cargill Incorporated Brazil) was added to a 12 L 4-neck round bottom flask. 22 g of Pure Flo B80 and 22 g of celite 545 were added to the reactor and the reactor was heated to 65° C. under nitrogen sparge. After 20 minutes of mixing at temperature the slurry was filtered using a Buchner funnel and celite coated filter paper.

6050 g of treated Soy FAME and 243 g of 88% formic acid were added to a clean 12 L 4-neck round bottom flask and heated to 55° C. under nitrogen sparge. Once at temperature, 2110 g of 70% hydrogen peroxide were added carefully over six hours to control the exothermic nature of the reaction. The reaction was maintained at 55° C. until the iodine value was <3 cg I/g. After 14 hours the iodine value was <3 cg I/g, the reaction was cooled to room temperature and agitation was stopped to allow for phase separation. The aqueous layer was decanted and the organic layer was washed 1 time with a 4% sodium bicarbonate solution and 2 times with water, 2000 g each. After the third wash and decant the epoxidized soy FAME was heated to 115° C. and vacuum of 5 Torr was applied to lower the peroxide value and dry the material. After 2 hours at 115° C. and 5 Torr the resulting product was cooled and analyzed. This resulted in product with an acid value of 1 mg KOH/g, a hydroxyl value of 27.8 mg KOH/g, an epoxy oxygen content of 6.41%, an iodine value of 2.1 cg I/g, and an APHA color of 78.

2838 g of the epoxidized soy FAME was added by addition funnel over 7 hours to a 2 inch Pope wiped film evaporator operating at 115° C., 0.2 torr, and a wiper speed of 414 rpm. The yield was 74% residue and 26% Distillate. The resulting product had 2.4% total saturates, an acid value of 0.95 mg KOH/g, epoxy oxygen content of 7.4% and a hydroxyl value of 35 mg KOH/g, 0.4% MAG, and an APHA color of 140.

Example 1B (Low saturates—High MAGs) (Saturates 2.2%—MAGs 3.3%) was prepared as follows. Material from Example 1D was added to a wiped film evaporator as in Example 1A under the same conditions to yield a product with an acid value of 0.8 mg KOH/g, epoxy oxygen content of 8.3%, hydroxyl value of 22 mg KOH/g, APHA color of 46, and 3.3% residual MAGs and Saturates of 2.2%.

Example 1C (High saturates—Low MAGs) (Saturates 16%—MAGs 0.4%) Example C is a commercially available B100 Soy FAME from Cargill Incorporated and was epoxidized as in Example 1A above.

Example 1D (High saturates—High MAGs) (Saturates 15%—MAGs 2.4%) Example 1D was prepared by the following procedure. Epoxidized Soybean Oil (commercially available from ASC as EPOXOL 7-4) is transesterified by methods well known in the art. Epoxidized soybean oil is heated to 65° C. to 70° C. with a molar excess of methanol in the presence of sodium methoxide as a basic catalyst. After equilibrium is reached, the reaction is neutralized with phosphoric acid solution and the glycerin by-product is decanted. Any excess methanol or moisture is removed with gentle heat under vacuum yielding a product with an acid value of 0.8 mg KOH/g, hydroxyl value of 16 mg KOH/g, Epoxy oxygen content of 7%, APHA color of 30, and 2.4% residual MAG.

Test plasticizer samples with varying contents of saturates and MAGs were prepared by blending Examples 1A-1D in various ratios. For example, samples were prepared from 75% 1A and 25% 1B; 50% 1A and 50% 1B; and 25% 1A and 75% 1B. These plasticizer and plasticizer blends were then used to prepare text plaques.

Test plaques were prepared from the following test formulas for both non-filled and filled systems.

TABLE 1

| Component | (Non-Filled System)* | (Filled System)* |
|---|---|---|
| PVC Resin (K71) | 100 | 100 |
| Norvic SP1300FA | | |
| Test Plasticizer or Plasticizer | 66.4 | 66.4 |
| Blend | | |
| Ca/Zn Stabilizer | 2 | 2 |
| BBC Stavin 1808 | | |
| CaCO₃ | | 100 |
| Huber Hubercarb G35 | | |
| Carbon Black | 0.41 | 0.41 |
| Raven L - Birla Carbon | | |
| | Physical Properties | |
| Plaque Shore Hardness | 60A +/− 2A | 75A +/− 2A |
| Plaque Weight | ~40 g | ~60 g |

*Values in Table are represented as PHR.

Small batch dry blends were made in 32 oz lab cups. The components of each recipe were added and mixed with a hand held mixer on a drill. The wet samples were placed in a 70° C. convection oven for 30 minutes, then mixed by hand to ensure the sample had become substantially or completely dry and free flowing.

The dry blends were then compounded with a two-roll mill at 160° C. for a total of 3 minutes, resulting in a sheet of PVC ˜1.5 mm in thickness.

The sheet was then cut into small squares of PVC that would be pressed into a plaque using a hydraulic press. Samples were placed between two mirror plates in a mold with a cavity size of 150 mm×150 mm×1.7 mm. Each sample would initially be placed in a set of heating plates, then the sample would be removed and placed in a set of cooling plates. The press conditions for each stage are as follows: (i) Heating—Plates set to 190° C. Prepress set to 60 seconds @4 MPa/Press set to 180 seconds @12 MPa (4-minute heating duration; (ii) Cooling—Plates set to 20° C. Prepress set to 5 seconds @4 MPa/Press set to 235 seconds @12 MPa (4-minute cooling duration). After pressing, the sample would be removed from the press have excess material trimmed, resulting in a square PVC plaque used for testing.

The test plaques were evaluated for susceptibility to crystallization by temperature cycling. The prepared plaques were placed onto a sheet of wax paper inside of a 6″×6″×4″ box. Boxes were dated and the samples were exposed to a temperature cycling. Overnight, the samples would be aged in a 5° C. fridge. During the day, the samples would be aged at room temperature. Approximately 12 hours at 5° C. followed by 12 hours at RT. Samples were aged for a total of 30 days and visually inspected daily and rated on a scale from 1-5. A value of 1 means no crystal or clear plaque. A value of 2 means a few small crystals are observed only in portions of the plaque surface. A value of 3 means small crystals are present across all portions of the plaque surface. A value of 4 means larger crystals are present across all portions of the plaque surface. A value of 5 means the surface of the plaque is completely covered in crystals and appears foggy. For sake of evaluation a value of 1 would be considered passing, a value of 2 would be marginal, and a value of 3-5 would be a failure.

### Example 2

Plaques were prepared as described above to maintain constant saturates level with increasing MAGs. As shown in Table 2, crystallization was clearly observed as MAG content increased. Results were the same for both filled and non-filled systems.

TABLE 2

| | Example | (1A/1B) | (1A/1B) | (1A/1B) | Example |
|---|---|---|---|---|---|
| | 1A | (75/25) | (50/50) | (25/75) | 1B |
| Sat % in | 2 | 2.05 | 2.1 | 2.15 | 2.2 |
| plasticizer | | | | | |
| MAG % in | 0.5 | 1.2 | 1.9 | 2.6 | 3.3 |
| plasticizer | | | | | |
| Crystallization | Pass | Pass | Marginal | Fail | Fail |

### Example 3

Plaques were prepared as described above to evaluate the effect of low MAG level being held constant and saturates levels increased. As shown in Table 3, no crystallization was observed (even after 30 days) in any of the samples despite dramatic increase in saturates level.

TABLE 3
Non Filled System.

| | Example | (1A/1C) | (1A/1C) | (1A/1C) | Example |
|---|---|---|---|---|---|
| | 1A | (75/25) | (50/50) | (25/75) | 1C |
| Sat % in | 2 | 5.5 | 9 | 12.5 | 16 |
| plasticizer | | | | | |
| MAG % in | 0.5 | 0.475 | 0.45 | 0.425 | 0.4 |
| plasticizer | | | | | |
| Crystallization | Pass | Pass | Pass | Pass | Pass |

### Example 4

Plaques were prepared as described above to evaluate the effect of saturates being held constant at higher level with increasing MAGs content. As shown in Table 4 and consistent with the observation in Example 2, crystallization was observed as MAG content increased.

TABLE 4
Non Filled System

| | Example | (1C/1D) | (1C/1D) | (1C/1D) | Example |
|---|---|---|---|---|---|
| | 1C | (75/25) | (50/50) | (25/75) | 1D |
| Sat % in | 16 | 15.75 | 15.5 | 15.25 | 15 |
| plasticizer | | | | | |
| MAG % in | 0.4 | 0.925 | 1.45 | 1.975 | 2.5 |
| plasticizer | | | | | |
| Crystallization | Pass | Pass | Pass | Marginal | Fail |

### Example 5—Permanence Evaluation

Test plaques were also prepared for evaluation of Permanence. The term “Permanence” as used herein mean the total mass loss during the heat aging of a plaque. Mass loss can be caused by loss of volatile organic compounds (VOC) or through exudation. Exudation is the migration of liquid or oily components to the surface of a plaque and represents a breakdown of the PVC/plasticizer structure.

Each plaque was weighed prior to heat aging. The samples were then hung in the oven and heat aged for 7 days at 50° C. Upon completion of aging, samples were weighted immediately upon removal from oven. This gives VOC mass loss (Initial Weight−Post Aging Weight=Weight Loss due to VOC). Samples were then wiped using a paper towel wetted with heptane, after allowing to dry the samples were weighed again. The change in mass is characterized as loss due to exudation. Total mass-loss (VOC+exudation) reflects plasticizer's permanence.

As shown in Table 5, reduction of saturates in the plasticizer dramatically impacts the level of VOCs given off by a sample upon aging. For example, VOC loss of Example 1C (16% sats) is 17 times greater than that of Example 1A (2% sats).

TABLE 5
Non Filled System (*After aging 7 days at 50 C.)

| | Example | (A/C) | (A/C) | (A/C) | Example |
|---|---|---|---|---|---|
| | A | (75/25) | (50/50) | (25/75) | C |
| Sat % in | 2 | 5.5 | 9 | 12.5 | 16 |
| plasticizer | | | | | |
| MAG % in | 0.5 | 0.475 | 0.45 | 0.425 | 0.4 |
| plasticizer | | | | | |
| VOC Loss* | 0.050 | 0.38 | 0.64 | 0.77 | 0.87 |
| Exudation* | 0.032 | 0.018 | 0.026 | 0.022 | 0.035 |

1. A plasticizer composition comprising an epoxidized fatty acid C1-C6 alkyl ester component wherein the epoxidized fatty acid C1-C6 alkyl ester component comprises (i) less than 6% saturated fatty acid residues, and (ii) between 0.1% and 2% monoacylglycerols.

2. The composition of claim 1 comprising greater than 90% epoxidized fatty acid C1-C6 alkyl esters or between 90% and 99.5% epoxidized fatty acid C1-C6 alkyl esters.

3. The composition of claim 1 comprising less than 3% saturated fatty acid residues.

4. The composition of claim 1 comprising between 0.5% and 3% saturated fatty acid residues.

5. (canceled)

6. The composition of claim 4 comprising between 0.1% and 1.0% monoacylglycerols.

7. The composition of claim 1 wherein the epoxidized fatty acid C1-C6 alkyl ester component comprises less than 2.5% C20 or greater fatty acid residues.

8. The composition of claim 1 wherein the epoxidized fatty acid C1-C6 alkyl ester component comprises between 0.1 and 2.5% C20 or greater fatty acid residues.

9. The composition of claim 1 wherein the epoxidized fatty acid C1-C6 alkyl ester component is a methyl ester.

10. The composition of claim 4 wherein the epoxidized fatty acid C1-C6 alkyl ester component is a methyl ester.

11. The composition of claim 10 wherein the epoxidized fatty acid C1-C6 alkyl ester component is a methyl ester derived from soybean oil.

12. A plasticized polymer composition, comprising (a) 100 parts by weight of at least one polymer resin; and (b) about 10 to 150 parts by weight of a plasticizer compounded with the at least one polymer resin, wherein the plasticizer comprises an epoxidized fatty acid C1-C6 alkyl ester component wherein the epoxidized fatty acid C1-C6 alkyl ester component contains (i) less than 7% saturated fatty acid residues and (ii) 0.1 to 2% monoacylglycerols.

13. The plasticized polymer composition of claim 12 where in the polymer resin is PVC.

14. A plasticized polymer composition of claim 12 wherein epoxidized fatty acid C1-C6 alkyl ester component is comprised of: a) between 1% and 3% saturated fatty acid residues; and b) between 0.1% and 0.5% monoacylglycerols.

15. A plasticized vinyl chloride composition of claim 11 further comprising an epoxidized natural oil.

16. A plasticized vinyl chloride composition of claim 11 further not comprising an epoxidized natural oil.

17. A process to prepare a plasticizer comprising the steps of a) providing a fatty acid C1-C6 alkyl ester component having less than 2% monoacylglycerols; b) epoxidizing the fatty acid C1-C6 alkyl ester component to yield an epoxidized fatty acid C1-C6 alkyl ester component; and c) removing the saturated fatty acid C1-C6 alkyl esters in the epoxidized fatty acid C1-C6 alkyl ester component to level below 7%, and d) recovering the plasticizer.

18. The plasticized polymer composition of claim 12 comprising polylactic acid.

19. The plasticized polymer composition of claim 12 which is substantially free of dioctyl phthalate (DOP), di-isononyl phthalate (DINP), dioctyl terephthalate (DOTP) and/or dioctyl cyclohexanoate (DOCH).
